(19) United States
(12) Patent Application Publication
(10) Pub. No.: US 2009/0048289 A1
(43) Pub. Date: Feb. 19, 2009

(54) ADMINISTRATION OF TROPISETRON FOR TREATING INFLAMMATORY SKIN DISEASES/DISORDERS

(75) Inventors: NADEGE TREMEL, Valbonne (FR); JEAN-DOMINIQUE PIERRET, Valbonne (FR); FABRIZIO DOLFI, Valbonne (FR); CHRISTIAN LOESCHE, Valbonne (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 12/193,362

(22) Filed: Aug. 18, 2008

Related U.S. Application Data
(63) Continuation of application No. PCT/EP2007/051778, filed on Feb. 23, 2007.

(30) Foreign Application Priority Data
Mar. 1, 2006 (EP) 06290348.9

(51) Int. Cl. A61K 31/46; A61P 17/00

(52) U.S. Cl. 514/304

(57) ABSTRACT

Tropisetron or salt thereof, formulated into diverse pharmaceutical/dermatological compositions, is useful for the treatment of a variety of inflammatory skin disorders/diseases, e.g., eczema, psoriasis, rosacea, acne vulgaris, ulcers, seborrhoeic dermatitis and irritations induced by chemical, physical or mechanical agents, notably rosacea.

### CROSS-REFERENCE TO PRIORITY/PCT APPLICATIONS

This application claims priority under 35 U.S.C. § 119 of EP 06290348.9, filed Mar. 1, 2006, and is a continuation/national phase of PCT/EP 2007/051778, filed Feb. 23, 2007 and designating the United States (published in the English language on Sep. 7, 2007 as WO 2007/099069 A1), each hereby expressly incorporated by reference in its entirety and each assigned to the assignee hereof.

### BACKGROUND OF THE INVENTION

1. Technical Field of the Invention

The present invention generally relates to the formulation of tropisetron as an active principle into pharmaceutical compositions, more particularly dermatological compositions, useful for the treatment of inflammatory skin disorders/diseases, and particularly for the treatment of rosacea.

2. Description of Background and/or Related and/or Prior Art

Tropisetron, or (±) 1H-Indole-3-carboxilic acid (3-endo)-8-methyl-8-azabicyclo[3.2.1]oct-3-yl-ester, is a compound which is already known per se, and is disclosed and claimed in U.S. Pat. No. 4,789,673. Tropisetron acts as a potent and selective antagonist of the neuronal 5HT receptor type 3, found associated with neurones of both peripheral (for example, on sensory nerve endings in the skin) and central origin. Specifically, tropisetron has been approved in the past in palliative therapy to prevent or treat chemotherapy induced nausea and vomiting and to treat post-operative nausea and vomiting.

In addition, this compound has recently been shown as being useful as an analgesic an anti-inflammatory agent: for instance, in vivo data obtained by Müller and Stratz who found tropisetron to be potent inhibitor of inflammation diseases such as rheumatic arthritis in “Local treatment of tendinopathies and myofascial pain syndrome with the 5-HT3 receptor antagonist tropisetron” Müller W et al.—Scand. J. Rheumatol., 2004; 119:44-48.

More broadly in “Physiology and pathophysiology of the 5-HT3 receptor” (Farber L et al.—Scand. J. Rheumatol. Suppl., 2004; (119):2-8), effects of 5-HT3 receptor antagonists have been described in the following disorders: intestinal effects (particularly in IBS and diarrhoea treatment); cardiac effects; central nervous system related disorders like anxiety, drug addiction, cognitive functions and thermoregulation, in nociception and pain particularly with rheumatoid arthritis, tendinopathies and myofascial pain as well as long-lasting analgesic and anti-phlogistic effect (by local or intra-articular injection), chronic fatigue syndrome and certain forms of pruritus.

There is, however, no published data regarding the use of tropisetron in order to treat an inflammatory skin disorder/disease and preferably rosacea.

### SUMMARY OF THE INVENTION

Thus, the present invention features the administration of an effective amount of at least one compound selected from among tropisetron and salts thereof for the treatment and/or prevention of inflammatory skin disorders and/or inflammatory skin diseases, particularly inflammatory skin disorders and/or skin diseases induced by the presence of a pathogenic agent (for example virus, bacterian or genetic agent) and preferentially for the treatment of rosacea, preferably via the oral or topical routes.

By “effective amount” is meant a sufficient amount to prevent and/or treat inflammatory skin disorders and/or skin diseases, and particularly to treat rosacea, by the oral or topical routes.

Additionally, the present invention features a novel regime or regimen of treating inflammatory skin disorders and/or skin diseases, particularly to treat inflammatory skin disorders and/or skin diseases induced by the presence of a pathogenic agent and preferentially to treat rosacea, preferably by the topical or oral routes, by topically applying or orally administering a pharmaceutical composition comprising tropisetron or salt thereof.

By “salts” of tropisetron is intended its ionic salts, and preferably its hydrochloride salt.

For the purpose of the instant invention, the inflammatory skin disorders and/or skin diseases induced by the presence of a pathogenic agent are understood as being at least partially caused by or resulting from or being the skin manifestation(s) of the presence of a living pathogen such as virus, yeast, fungus, protozoon, bacterial agent or genetic alteration.

Among other causes, rosacea is for example due to the pathogen Demodex folliculorum. Likewise, acnea, and notably acnea vulgaris, is due to Propionibacterium acnes.

### DETAILED DESCRIPTION OF BEST MODE AND SPECIFIC/PREFERRED EMBODIMENTS OF THE INVENTION

Rosacea is a common, but often overlooked, skin condition of uncertain etiology that can lead to significant facial disfigurement, ocular complications, and severe emotional distress. A reviewed standard classification system for rosacea was published in the June 2004 issue of the Journal of the American Academy of Dermatology. Developed by the National Rosacea Society Expert Committee on the Classification and Staging of Rosacea and reviewed by rosacea experts worldwide, it describes primary and secondary features of rosacea (as described below) and recognizes 4 patterns of signs and symptoms, designated as subtypes:

Subtype 1: Erythematotelangiectatic Rosacea:

Subtype 1 is characterized by flushing and persistent central facial erythema. Telangiectases are common but not essential for the diagnosis.

Subtype 2: Papulopustular Rosacea:

Subtype 2 includes persistent central facial erythema with transient papules, pustules, or both in a central facial distribution. Burning and stinging may also be reported.

Subtype 3: Phymatous Rosacea:

This subtype may include thickening skin, irregular surface nodularities, and enlargement. Phymatous rosacea occurs most commonly as rhinophyma but may appear elsewhere, including the chin, forehead, cheeks, and ears. Patulous, expressive follicles may appear in the phymatous area, and telangiectases may be present.

Subtype 4: Ocular Rosacea:

Ocular rosacea may include watery or bloodshot appearance (interpalpebral conjunctival hyperemia), foreignbody sensation, burning or stinging, dryness, itching, light sensitivity, blurred vision, telangiectasia of the conjunctiva and lid margin, or lid and periocular erythema. Blepharitis, conjunctivitis, and irregularity of the eyelid margins also may occur. Meibomian gland dysfunction presenting as chalazion, or chronic infection as manifested by hordeolum (stye), are common. Some patients may experience loss of vision as a result of corneal complications (punctate keratitis, corneal infiltrates, ulcers, or marginal keratitis). An ophthalmologic consultative approach to treatment may be required.

Central facial erythema and telangiectasis are predominant in the early stages of rosacea. This progresses to a chronic inflammatory infiltrate with central facial papules and pustules. Intermittent or chronic facial edema may also occur. Some patients develop rhinophyma, a coarse hypertrophy of the connective tissue and sebaceous glands of the nose evident as small knobby bumps on the nose.

The predominant, presenting complaints of rosacea are intermittent, central facial flushing and erythema. Itching is typically absent; however, many patients complain of a stinging pain (which can be severe) associated with flushing episodes. These flushing episodes are often socially embarrassing and can occur unpredictably or can be linked to environmental, chemical, food, or emotional triggers. Common triggers include exposure to the sun, cold weather, sudden emotion (laughter or embarrassment), hot beverages, and alcohol consumption.

According to the present invention, tropisetron is administered by any routes, i.e., orally enterally, parenterally (subcutaneously, cutaneously, intravenously, intramuscularly), topically or ocularly, but preferentially as mentioned above by oral, topical or ocular routes.

Therefore, topical tropisetron (in cream, gel, or lotion form, for example) or oral tropisetron (in tablets, capsules, powder, syrup or liquid forms for instance) administered once or twice daily is effective for the treatment of rosacea subtypes, particularly early stages of rosacea and preferably facial flushing and erythema.

The present invention also relates that topical or oral tropisetron is appropriate to treat each subtype symptoms (disclosed previously) separately or in combination and hence can reduce pustular and papular rosacea, ocular rosacea and telangiectasis in certain patients.

As a consequence, this invention features the formulation of an effective amount of at least one compound selected from among tropisetron and salts thereof into pharmaceutical compositions for treating rosacea. This invention also features the formulation of tropisetron or salts thereof into topical pharmaceutical compositions for the treatment of all subtypes of rosacea and preferably for the treatment of early stages of rosacea, i.e., wherein the rosacea is treated at a stage in which facial flushing and skin redness is evident.

In another embodiment, the present invention also features the formulation of tropisetron or salts thereof into topical pharmaceutical compositions for the treatment of erythematotelangiectatic rosacea, papulopustular rosacea, phymatous rosacea and/or ocular rosacea.

Rosacea is a chronic, relapsing disorder, and a long-term treatment is generally required. Control of symptoms can be successfully maintained by topical or oral use of tropisetron.

Other characteristics, aspects, objectives and advantages of the invention will become still more clearly apparent from the description which follows and various specific, but in no way limiting, examples illustrating same.

In a preferred embodiment, the pharmaceutical compositions of the invention are useful for topical application. More particularly, the pharmaceutical composition is a dermatological composition.

Such a composition according to the present invention is a composition, in particular for the topical treatment of inflammatory skin disorders/diseases, comprising an effective amount of a compound selected from among tropisetron and its salts and a topical pharmaceutically acceptable carrier therefore.

By “topical pharmaceutically acceptable carrier” is meant a carrier compatible with the skin, hair and/or mucous membranes, which is applied topically.

The dermatological composition is more generally useful for the treatment, by the topical route, of skin diseases or complaints having at least a vascular lesion, one inflammatory component or, at the same time, one inflammatory and one infectious component.

More particularly, the skin diseases or disorders correspond to skin inflammations accompanying any type of dermatosis such as eczema, psoriasis, rosacea, acne vulgaris, ulcers, seborrhoeic dermatitis and irritations induced by chemical, physical or mechanical agents or others. The present invention is especially useful in the topical treatment of rosacea using tropisetron or its salts.

In what follows, “topical route” means any technique for administration of a product by direct application to a surface (or external) part of the body, such as the skin or eye.

By the topical route, the pharmaceutical compositions comprising tropisetron or its salts, which are therefore more particularly useful for the treatment of the skin or mucous membranes, can be provided in the form of ointments, creams, salves, powders, impregnated pads, solutions, gels, sprays, lotions or suspensions. They can also be provided in the form of lipid or polymer vesicles or nanospheres or microspheres or in the form of polymer patches or hydrogels making possible a controlled release formulation. These compositions by the topical route can moreover be provided either in anhydrous form or in an aqueous form, according to the clinical indication. Formulations for topical use which are particularly highly suitable according to the present invention are given in particular in the examples to follow.

In a preferred embodiment of the invention, the topical composition is in the form of a gel, a cream or a lotion.

By the ocular route, the compositions of the present invention are mainly eyewashes or eye drops.

For the purposes of the present invention, oral route is understood as any technique for administration of a product by digestive absorption.

By oral route, the compositions of the present invention are tablets, capsules, pills, coated-tablets, powders, granules, syrups, emulsions, solutions, suspensions as well as microspheres or nanospheres or lipidic or polymeric vesicles for slow or controlled-release.

The compositions, preferably for topical or oral use, according to the invention contain tropisetron or its salts at a concentration preferably of from 0.0001% to 20% by weight with respect to the total weight of the pharmaceutical composition, more preferably at a concentration ranging from 0.01% to 1%.

According to a specific and preferred embodiment of the present invention, the overall content of the tropisetron or its salts does not exceed 20% of the total weight of the composition. Preferably, the medicinal compositions are topically applied once or twice a day, for a period of a maximum of 3 months.

In addition, the said composition could also be used for a cosmetic use for alleviating skin aspect. In particular, said composition can improve the appearance of skin and/or skin radiance and/or skin roughness.

The medicinal compositions according to the invention can, of course, additionally contain inert or even pharmacodynamically or cosmetically active additives or combinations of these additives and in particular: wetting agents; depigmenting agents such as hydroquinone, azelaic acid, caffeic acid or kojic acid; emollients such as glycerine; hydrating agents such as glycerol, PEG 400, thiamorpholinone and its derivatives or, alternatively, urea; anti-seborrhoeic or anti-acne agents, such as S-carboxymethylcysteine, S-benzylcysteamine, their salts or their derivatives, or benzoyl peroxide; fungicides such as ketoconazole or 4,5-polymethylene-3-isothiazolidones; carotenoids and in particular beta-carotene; anti-psoriatic agents such as anthralin and its derivatives; and finally 5,8,11,14-eicosatetraynoic acid and 5,8,11-eicosatriynoic acid and their esters and amides; metronidazole, ivermectine, and other agents used for treating rosacea; anti-inflammatory agents such as NSAIDs (Non Steroidal Anti-Inflammatory Drugs).

The compositions according to the invention can also contain flavor-improving agents, preservatives, such as the esters of para-hydroxybenzoic acid, stabilizing agents, moisture-regulating agents, pH-regulating agents, agents for modifying osmotic pressure, emulsifying agents, UV-A and UV-B screening agents, and antioxidizing agents, such as alpha-tocopherol, butylated hydroxyanisole or butylated hydroxytoluene.

Of course, one skilled in this art will take care to choose the optional compound(s) to be added to the pharmaceutical composition so that the advantageous properties intrinsically attached to the composition are not, or not substantially, detrimentally affected by the envisaged addition.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Example 1

This example is of a specific formulation in accordance with the invention which is provided in the form of a gel for topical application:

| | |
|---|---|
| Tropisetron HCl | 0.75 g |
| Carbopol 980 (Goodrich) | 0.6 g |
| Polyethylene glycol 400 | 3 g |
| Sodium hydroxide | q.s. pH 5 |
| Preservatives | q.s. |
| Demineralized water | q.s. for 100 g |

### Example 2

This example is of a specific formulation in accordance with the invention which is provided in the form of a cream for topical application:

| | |
|---|---|
| Tropisetron HCl | 0.75 g |
| Methyl glucose sesquistearate | 1 g |
| Stearyl alcohol | 0.5 g |
| Liquid paraffin oil | 6 g |
| Polyethylene glycol 400 | 2 g |
| Methyl glucose sesquistearate | 5 g |
| polyoxyethylenated with 20 mol of EO | |
| Carbopol 981 (Goodrich) | 0.4 g |
| Glycerol | 7 g |
| Cyclomethicone | 4 g |
| Sodium hydroxide | q.s. pH 5 |
| Preservatives | q.s. |
| Demineralized water | q.s. for 100 g |

### Example 3

This example is of a specific formulation in accordance with the invention which is provided in the form of a lotion for topical application:

| | |
|---|---|
| Tropisetron HCl | 0.75 g |
| Benzyl alcohol | 1.30 |
| Glycerol | 7.00 |
| Stearyl alcohol | 2.00 |
| Light mineral oil | 6.00 |
| Carbomer 941 | 0.15 |
| Glyceryl Stearate | 3.00 |
| Potassium Sorbate | 0.20 |
| Cyclomethicone | 4.00 |
| PEG-8 | 2.00 |
| Steareth-21 | 3.00 |
| Lactic acid | q.s. pH adjustment |
| Sodium Hydroxyde | q.s. pH adjustment |
| Purified Water | q.s. 100.00 |

### Example 4

This example is of a specific formulation in accordance with the invention which is provided in the form of a blend powder for capsule for oral administration:

| | | |
|---|---|---|
| Tropisetron HCl | 5 | mg |
| Mannitol | 83.5 | mg |
| Corn starch | 10 | mg |
| Anhydrous colloidal Silica | 0.5 | mg |
| Magnesium Stearate | 1 | mg |

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for the treatment of an inflammatory skin disorder/disease, comprising administering to a subject in need of such treatment, a thus effective amount of tropisetron or salt thereof.

2. The regime or regimen as defined by claim 1, said inflammatory skin disorder/disease having been inducted by a pathogenic agent.

3. The regime or regimen as defined by claim 1, said inflammatory skin disorder/disease being selected from the group consisting of eczema, psoriasis, rosacea, acne vulgaris, ulcers, seborrhoeic dermatitis and irritations induced by chemical, physical or mechanical agents.

4. The regime or regimen as defined by claim 3, comprising the treatment of rosacea.

5. The regime or regimen as defined by claim 1, comprising topically applying said tropisetron or salt thereof onto the afflicted skin area of said subject in need of such treatment.

6. The regime or regimen as defined by claim 1, comprising orally administering said tropisetron or salt thereof to said subject in need of such treatment.

7. A regime or regimen for the treatment of an inflammatory skin disorder/disease, comprising administering to a subject in need of such treatment, a pharmaceutical/dermatological composition which comprises a thus effective amount of tropisetron or salt thereof, formulated into a pharmaceutically acceptable carrier therefor.

8. The regime or regimen as defined by claim 7, said pharmaceutical/dermatological composition comprising from about 0.0001% to 20% by weight of said tropisetron or salt thereof.

9. The regime or regimen as defined by claim 7, said inflammatory skin disorder/disease being selected from the group consisting of eczema, psoriasis, rosacea, acne vulgaris, ulcers, seborrhoeic dermatitis and irritations induced by chemical, physical or mechanical agents.

10. The regime or regimen as defined by claim 9, comprising the treatment of rosacea.

11. The regime or regimen as defined by claim 9, comprising the treatment of early stage rosacea.

12. The regime or regimen as defined by claim 9, comprising the treatment of erythematotelangiectatic rosacea.

13. The regime or regimen as defined by claim 9, comprising the treatment of papulopustular rosacea.

14. The regime or regimen as defined by claim 9, comprising the treatment of phymatous rosacea.

15. The regime or regimen as defined by claim 9, comprising the treatment of ocular rosacea.

16. The regime or regimen as defined by claim 7, comprising topically applying said pharmaceutical/dermatological composition onto the afflicted skin area of said subject in need of such treatment.

17. The regime or regimen as defined by claim 7, comprising orally administering said pharmaceutical/dermatological composition to said subject in need of such treatment.

18. The regime or regimen as defined by claim 16, said pharmaceutical/dermatological composition comprising a gel.

19. The regime or regimen as defined by claim 16, said pharmaceutical/dermatological composition comprising a cream.

20. The regime or regimen as defined by claim 16, said pharmaceutical/dermatological composition comprising a lotion.

21. The regime or regimen as defined by claim 7, comprising alleviating skin aspect.
